(19) United States
(12) Plant Patent Application Publication
(10) Pub. No.: US 2004/0045060 P1
(43) Pub. Date: Mar. 4, 2004

(54) PISTACHIO ROOTSTOCK NAMED 'P.N. 1-15'

(76) Inventors: Henry P. Anderson III, Tulare, CA (US); Kenneth L. Puryear, Visalia, CA (US)

Correspondence Address: CHRISTIE, PARKER & HALE, LLP, P.O. BOX 7068, PASADENA, CA, 91109-7068, US

(21) Appl. No.: 10/232,050

(22) Filed: Aug. 30, 2002

(51) Int. Cl. A01H 5/00

(52) U.S. Cl. PLT/152

(57) ABSTRACT

A new and distinct variety of pistachio tree, characterized by the glossy, medium red to reddish green color of young foliage, the dull, dark green color of mature foliage, and the rounded upright growth habit, which is particularly useful as a rootstock upon which commercial nut bearing pistachio varieties may be budded or grafted.

### LATIN NAME OF GENUS AND SPECIES

The present invention relates to a new Pistachio sp. tree, designated ‘P.N. 1-15’.

### BACKGROUND OF THE INVENTION

The present invention relates to a new and distinct variety of pistachio tree, which is a hybrid of P. atlantica and P. integerrima.

### SUMMARY OF THE INVENTION

The ‘P.N. 1-15’ variety is characterized by the rounded and upright growth of the plant, the glossy, medium red to reddish green color of young foliage, and the dull, dark green color of mature foliage.

The variety is particularly useful as a rootstock upon which commercial nut bearing pistachio varieties may be budded or grafted.

Asexual reproduction by budding onto P. integerrima at Kern County, California shows that the foregoing and other distinguishing characteristics come true to form and are established and transmitted through succeeding propagation. Other known forms of asexual reproduction for pistachios may also be employed.

### BRIEF DESCRIPTION OF ILLUSTRATIONS

The accompanying photographic illustrations show typical specimens of vegetative growth of the new variety, in color as nearly true as it is reasonably possible to make in a color illustration of this character.

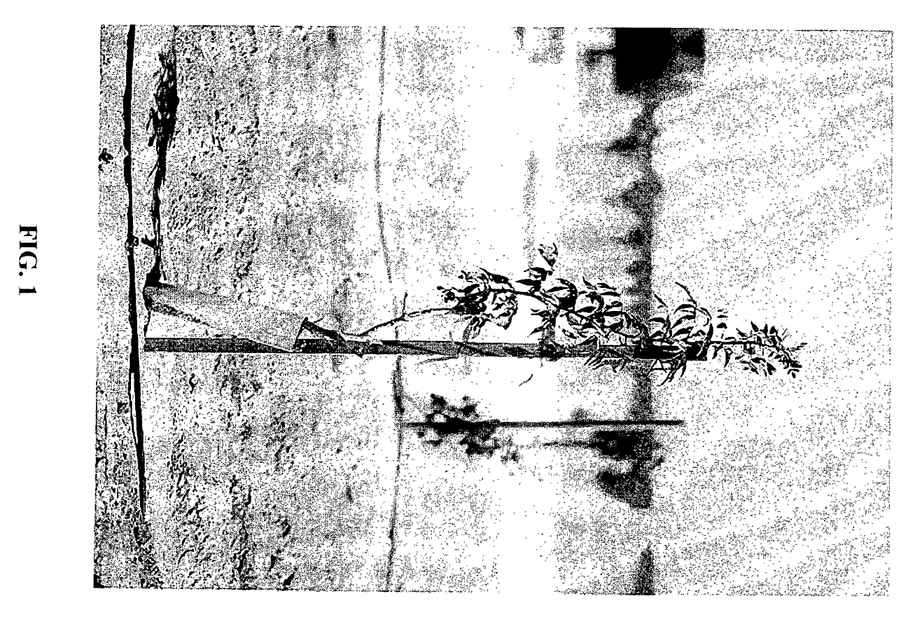

FIG. 1 shows a one year old pistachio tree of the new variety.

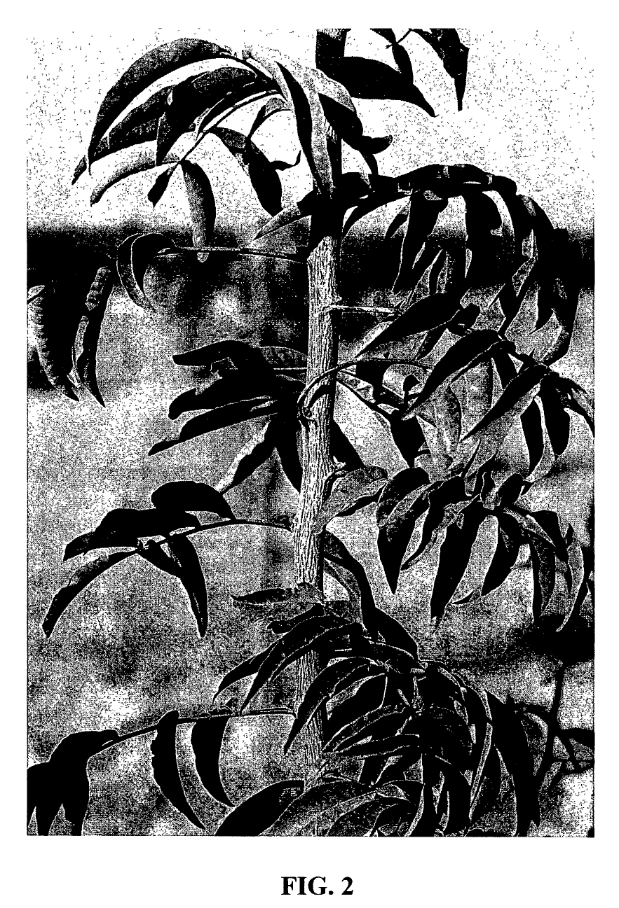

FIG. 2 shows a close-up of the trunk and compound leaves, typically with eleven leaflets.

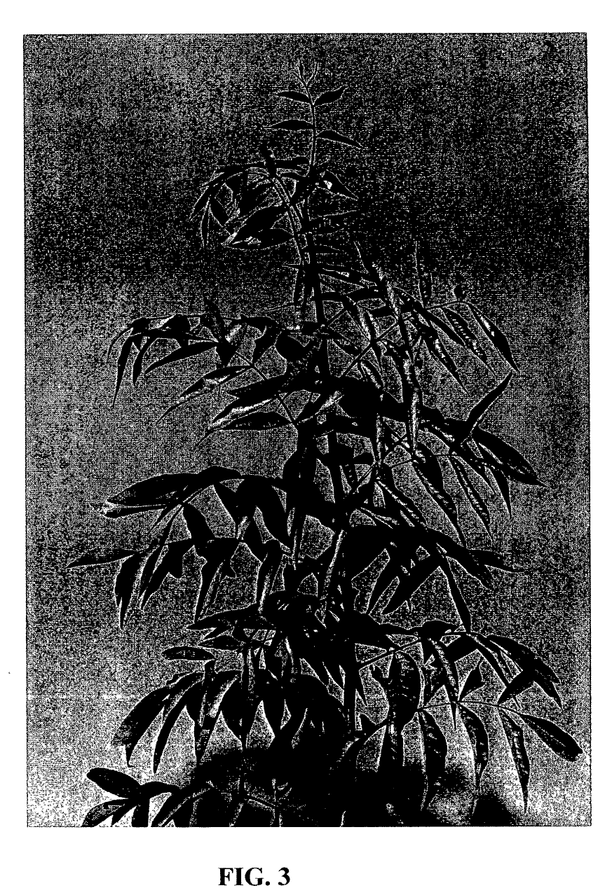

FIG. 3 shows a close-up of the new variety with both medium red to reddish green new leaflets and dark green older leaflets.

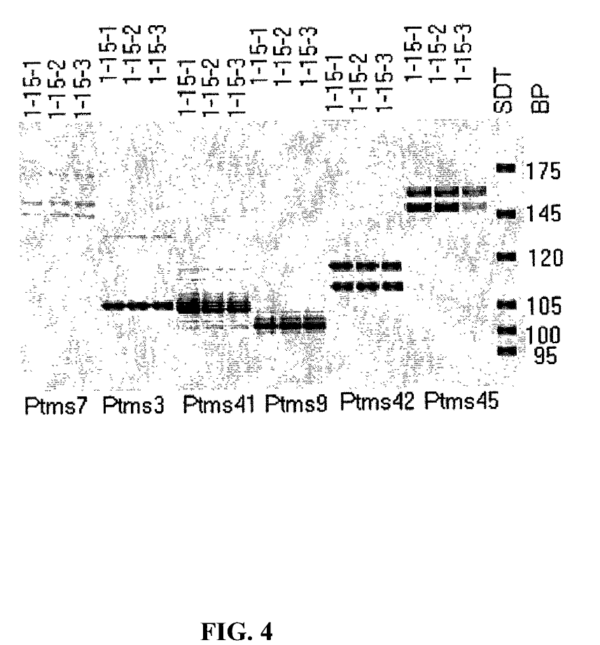

FIG. 4 shows an autoradiogram of a Southern blot, using various microsatellite DNA markers (Ptms7, Ptms3, Ptms41, Ptms9, Ptms42 and Ptms45) as probes directed against DNA isolated from ‘P.N. 1-15’. Each marker was run in triplicate. Size markers are shown in the lane labeled “SDT”.

### DESCRIPTION OF THE NEW VARIETY

Referring now specifically to the new and distinct variety of pistachio tree, the following is a detailed description of one year old trees with color description where indicated by reference to The Royal Horticultural Society Colour Chart, except where common terms of color definition are employed.

The new variety of pistachio tree as herein described may vary in slight detail due to climatic, soil and cultural conditions under which the variety may be grown, the present description being of the variety as grown at McFarland, Kern County, Calif.

Plant:

Growth.—Rounded and upright, as indicated in FIG. 1.

Foliage:

Leaves.—Compound of eleven leaflets (sometimes less), as indicated in FIG. 2.

Leaflets.—Small, with young tissue glossy and lighter green than older tissue, which is dull and darker green. Shape — Medium length and lanceolate, apex acute, base acute but less so than apex. Color — New leaflets are medium red to a reddish green, near Greyed-red Group 173A, as shown in FIG. 3. Older leaflets are dark green, near Greyed-green Group 191A, also as shown in FIG. 3.

Leaflet Ribs.—The center rib on leaves are deepened. Ribs of leaflets start at the base as deep red and progressively turn green at the tip of the leaflet. The veins in leaflets are lighter green than the leaflets.

Length of leaves.—The length of the leaves (including petiole and leaflets) averages about 7 inches long and about 4.5 inches to end of midrib. Individual leaflets average up to about 3 inches.

Growth:

Trunk.—Trunk is rounded.

Trunk color.—The trunk is light brown in color, near Greyed-orange Group 174B, on older growth and red, near Greyed-red Group 178A, in newer growth.

Vegetative buds.—Vegetative buds are small and reddish on new growth and green and larger on older growth.

We claim:

1. A new and distinct variety of pistachio tree, identified as ‘P.N. 1-15’, substantially as shown and described.
